(19) United States
(12) Patent Application Publication
(10) Pub. No.: US 2009/0131876 A1
(43) Pub. Date: May 21, 2009

(54) SHIELDABLE NEEDLE ASSEMBLY CONTAINING REDUCED STRESS LIVING HINGE AND METHODS OF MANUFACTURE

(76) Inventor: Martin M. Coyne, Allendale, NJ (US)

Correspondence Address: David W. Highet, VP & Chief IP Counsel;Becton, Dickinson and Company, 1 Becton Drive, MC 110, Franklin Lakes, NJ, 07417-1880, US

(21) Appl. No.: 12/090,470

(22) PCT Filed: Oct. 12, 2006
(86) PCT No.: PCT/US2006/039790

§ 371 Date: Oct. 16, 2008

Related U.S. Application Data
(60) Provisional application No. 60/727,487, filed on Oct. 17, 2005.

(51) Int. Cl. A61M 5/32; B23P 11/00; A61L 2/04

(52) U.S. Cl. 604/198; 29/428; 422/22

(57) ABSTRACT

A shieldable needle assembly is provided containing a shield with one or more reduced stress living hinges (10) which remain in the folded position. Methods of removing substantially all of the internal stress in the living hinge of a needle shield are also provided comprising irradiating and/or heat treating the living hinge.

### FIELD OF THE INVENTION

This invention relates generally to safety devices for hollow bore medical needles and particularly to syringe needle devices which employ protective needle shields or sheaths for securely shielding sharp needle tips, both before and after being used in a medical procedure.

### BACKGROUND OF THE INVENTION

Problems associated with inadvertent needle sticks are well known in the art of blood sampling, percutaneous medication injection and other medical procedures involving the use of medical needles. Ever increasing attention is being paid to needle stick problems due to the contemporary sensitivity of exposure to human immunodeficiency virus (HIV), hepatitis and other serious blood-borne diseases.

Commonly, procedures involving removing a needle from a patient require a technician to use one hand to place pressure at the wound site where the needle is being withdrawn while removing the needle apparatus with the other hand. It is common practice for a tending technician to give higher priority to care for the wound than is given to disposal of a needle. In the case of commonly used, non-safety devices, such priority either requires convenience of an available sharps container within ready reach or another means for safe disposal without leaving the patient's side. Providing adequate care, with accompanying safety procedures, is often compounded by patient physical condition and mental state (e.g., in burn units and psychiatric wards). Under such conditions, it is often difficult, if not impossible, to take appropriate action to properly dispose of a used, exposed needle while caring for a patient. Further, common practice of filling syringes with medication in one area and then transporting an uncapped needle (recapping a needle is currently discouraged in U.S. medical practice due to dangers associated with recapping) to a patient area provides a significant opportunity for accidental needle sticks.

Widespread knowledge and history associated with needle care and disposal problems have resulted in the conception and disclosure of a large number of devices, each of which represents an attempt to provide not only a solution to the problem of needle sticks, but also a device which is commercially viable (i.e., cost and price competitive with currently used non-safety devices). One such type of device employ a means for extending a protective safety shield over the needle immediately after use. Many of these devices employ one or more living hinge mechanisms to provide articulation arm segments that control the position of the shield. Movement of the arm segments from a retracted to extended position translates into movement of the shield from a proximal “needle tip exposed” position to a distal “needle tip enclosed” position. See, e.g., U.S. Pat. No. 5,348,544, U.S. Patent Publication No. 2001/0039401.

Living hinges can be provided by tethering together two larger pieces of plastic with a thin section of plastic. When integrally molded, the two larger sections can be hinged so they will bend relative to each other through the thinner section. Such living hinges are particularly advantageous for molded parts since they are a single molded section, and require no expensive tooling such as might be required to produce pinned or other types of hinge joints. For simplicity and efficiency in molding, it is generally easier to mold living hinges in a flat primary position and then subsequently fold them into a secondary position.

A drawback of these living hinge devices, however, is that they generally require mechanical retention (e.g., spring, latch, hook, detent, etc.) to overcome the stored energy and internal stress of the folded living hinge and maintain the shield in the needle tip exposed position. See, e.g., U.S. Pat. No. 5,348,544, U.S. Pat. No. 6,254,575, and U.S. Patent Publication No. 2001/0039401. It would be desirable to provide a reduced stress living hinge, for example, a shieldable needle assembly that does not require mechanical retention means for maintaining the shield in the needle tip exposed position.

### SUMMARY OF THE INVENTION

One or more embodiments of the present invention provides a shieldable needle assembly comprising a safety shield mounted on or near a needle cannula. The safety shield can be moved from a first position, where the tip of the needle cannula is exposed for use, to a second position where the tip of the needle cannula is substantially covered or shielded. According to one or embodiments, the shieldable needle assembly also includes one or more reduced stress living hinges which allow the safety shield to remain in the needle tip exposed first position in the absence of any mechanical retention, for example, a spring, a latch, a hook, a detent, etc.

Accordingly, one aspect of the present invention is directed to a shieldable needle assembly comprising: a needle cannula having a proximal end and a distal tip; a safety shield movable between a first position in which the distal tip of the needle is exposed and a second position configured to substantially enclose the distal tip of said needle cannula; and one or more reduced stress living hinges which maintain said safety shield in the needle tip exposed first position in the absence of mechanical retention.

One embodiment of the present invention is directed to a shieldable needle assembly comprising: a needle cannula securely engaged with a needle hub, said needle cannula having a proximal end and a distal tip; and a safety shield having a proximal end and a distal end, said shield further having a needle guard slidably moveable along said needle cannula and a distal segment and a proximal segment connected via a hinge, said proximal segment and said needle hub connected via a hinge and said distal segment and said needle guard connected via a hinge, said distal segment, said proximal segment and said needle guard being extensible between a retracted first position and an extended second position configured to substantially enclose the distal tip of said needle cannula in the second position, wherein one or more of said hinges is a reduced stress living hinge such that said distal segment, said proximal segment and said needle guard are maintained in the retracted needle tip exposed first position in the absence of mechanical retention.

Another embodiment of the present invention is directed to a shieldable needle assembly comprising: a needle cannula securely engaged with a needle hub, said needle cannula having a proximal end and a distal tip; and a safety shield having a proximal end and a distal end, said shield further having a distal segment and a proximal segment connected via a hinge, said proximal segment and said needle hub connected via a hinge, said distal segment and said proximal segment being extensible between a retracted first position and an extended second position in which the needle is enclosed, said distal segment slidably moveable along said needle cannula and configured to substantially enclose the distal tip of said needle cannula in the extended second position, wherein one or both of said hinges is a reduced stress living hinge such that said distal segment and said proximal segment are maintained in the retracted needle tip exposed first position in the absence of mechanical retention.

Another embodiment of the present invention is directed to a shieldable needle assembly comprising: a needle cannula securely engaged with a needle hub, said needle cannula having a proximal end and a distal tip; and a safety shield having a shield body, said shield body having a cover and a mount connected via a hinge, said mount mounted to said hub, said cover moveable between a proximal first position and a distal second position configured to substantially enclose the distal tip of said needle cannula in the distal second position, wherein said hinge is a reduced stress living hinge such that said cover is maintained in the proximal needle tip exposed first position in the absence of mechanical retention.

Another aspect of the invention pertains to a method of making a shielded needle assembly. According to one or more embodiments, the method comprises providing a foldable shield having at least one living hinge between two folded segments, the living hinge exhibiting internal stress such that the folded segments are urged to an open or flat configuration, assembling the shield to a device including a needle cannula including a distal tip such that one of the folded segments is configured to cover the distal tip of the needle, exposing at least the living hinge to heat and/or irradiation sufficient to remove substantially all of the internal stress in the living hinge.

These and other features of the present invention will be apparent from the detailed description taken with reference to accompanying drawings.

### BRIEF DESCRIPTION OF THE DRAWINGS

FIG. 1 shows the preparation of a reduced stress living hinge during application of force “F”;

FIG. 2 is a perspective view of a shieldable needle assembly according to an embodiment of the present invention having one or more reduced stress living hinges with the shield in a retracted needle tip exposed position;

FIG. 3 is a cross-sectional side view taken along line 2-2 in FIG. 2 with application of distally directed pressure to urge the shield to an extended needle tip enclosed position;

FIG. 4a is a perspective view of an alternative embodiment of the shieldable needle assembly in FIGS. 2 and 3;

FIG. 4b is a cutaway view of a locking mechanism used with the shieldable needle assembly in FIG. 4a; and

FIG. 5 is a cross-sectional side view of a shieldable needle assembly according to another embodiment of the present invention having a reduced stress living hinge with the shield in a proximal needle tip exposed position showing application of upwardly directed pressure.

FIG. 6 is a cross-sectional side view of the shieldable needle assembly in FIG. 5 with the shield in a distal needle tip enclosed position.

### DETAILED DESCRIPTION OF THE INVENTION

Before describing several exemplary embodiments of the invention, it is to be understood that the invention is not limited to the details of construction or process steps set forth in the following description. The invention is capable of other embodiments and of being practiced or carried out in various ways.

One aspect of the present invention is directed to a shieldable needle assembly comprising: a needle cannula having a proximal end and a distal tip; a safety shield movable between a needle tip exposed first position and a needle tip enclosed second position and configured to substantially enclose the distal tip of said needle cannula in the needle tip enclosed second position; and one or more reduced stress living hinges in the hinges have substantially no internal stress. According to one or more embodiments, the shield having such a reduced stress living hinges can maintain said safety shield in the needle tip exposed first position in the absence of mechanical retention, such as, for example, a spring, a latch, a hook, a detent, etc.

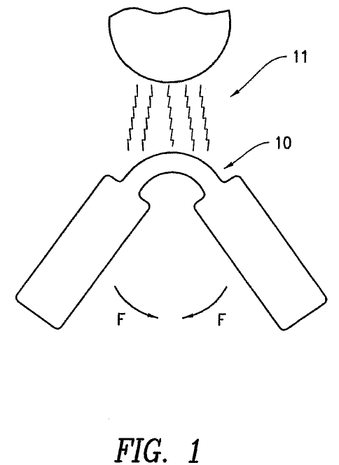

As used herein, the term “proximal” is generally used to indicate relative nearness of a referenced item to a user of a device or a viewer of a perspective drawing of a figure. The term “distal” is similarly used to indicate relative remoteness. As used herein, the term “first position” refers to when the needle shield or cover does not cover the needle tip. The term “second position” refers to when the needle shield or cover covers the needle tip. The terms “first position” and “second position” shall not be construed to exclude positions intermediate between the first position and second position. Reference is now made to the embodiments illustrated in FIGS. 1-5, wherein like numerals are used to designate like parts throughout.

FIG. 1 shows the preparation of a reduced stress living hinge. Thus, as used herein, “reduced stress living hinge” refers to a living hinge that exhibits substantially no tendency to move or spring towards a flat or unfolded condition. This can be determined by manufacturing a shield with a living hinge, processing the hinge to remove or reduced the stress in the hinge region and then empirically determining whether the hinge has a tendency to move towards a flat or unfolded condition. This can be accomplished by folding a shield including a hinge and applying force or a mechanical retention means to hold the shield in a folded condition. The force or mechanical retention means holding the shield in a folded condition can then be released, and the position of the portions of the shield attached to the hinge can be observed to determine if they move towards a flat or unfolded condition. According to embodiments of the present invention, the shield including the living hinge has been processed such that the living hinge exhibits less internal stress than a hinge that has been manufactured using normal manufacturing and sterilization processes, and in certain embodiments, the living hinge has substantially no internal stress. As used herein, a shield having a living hinge that has substantially no internal stress is one in which the portions of the shield joined by the hinge are not urged towards a flat or open configuration in the absence of force applied to the shield or mechanical retention means that hold the shield in a folded configuration. As used herein, the term “living hinge” refers to a hinge with no moving parts that bends to allow movement of an element attached to the hinge, regardless of the method of manufacture of the component including the living hinge.

Thus, in addition to the usual levels of irradiation or heat treatment of the device, additional heat or irradiation is applied for a longer time and/or at greater quantities or intensity to reduce the internal stress in the living hinge. In addition, the irradiation or heat treatment may be targeted or directed at the living hinge portion of the shield to reduce the stress therein. A shield including a reduced stress living hinge includes a hinge that does not require a mechanical retention means such as a latch to retain the shield in a folded condition.

The stress of the living hinge can be reduced using means well known to those skilled in the art, including but not limited to heat treatment and irradiation (e.g., Cobalt-60 or electron beam source) beyond the heat treatment or irradiation that is applied to sterilize the medical device. Referring now to FIG. 1 an example of a process for reducing or substantially eliminating the stress in a living hinge is shown. For example, energy 11 (e.g., heat or radiation) can be applied to a hinge 10 in the folded assembled position (e.g., while applying force “F”) for a time sufficient to relieve some or all of the stress induced in the folded hinge, thereby reducing or substantially eliminating the hinge's “memory” for the pre-folded position. This process can be integrated directly into the sterilization process, which for medical devices is a necessary step, and the duration and/or amount of radiation or heat applied to the hinge can be adjusted to obtain the desired amount of stress in the hinge. In addition, separate irradiation or heat treatments may be applied directly to the hinge area. Through basic laboratory testing (e.g., stress tests), process parameters (e.g., time, temperature, radiation levels, etc.) can be identified for any living hinge in a specific assembled situation that relieve the induced stress to a desired point. Preferably, the folded hinge is heated or irradiated such that substantially all of the stress in the folded hinge is relieved such that the hinge does not urge the shield towards an open configuration. The stress relief reduces the tendency of the living hinge to flex back towards its pre-folded position, thereby eliminating the need for mechanical retention means to maintain the hinge in the desired folded position.

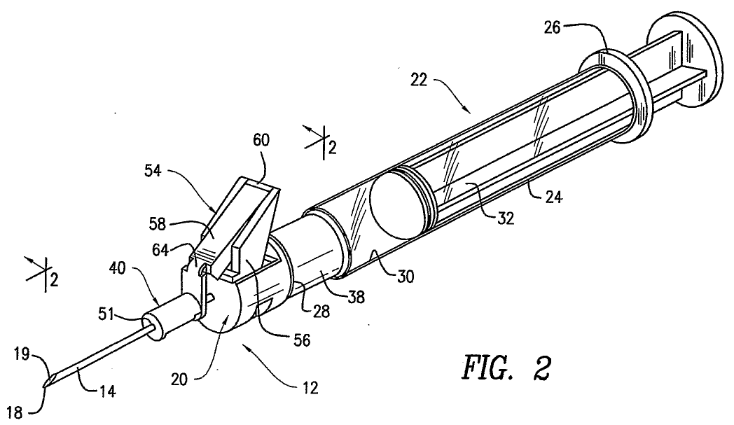

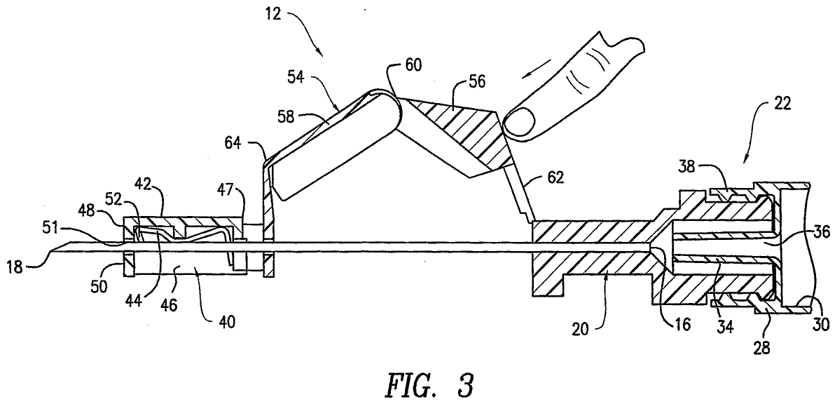

One embodiment of a shieldable needle assembly incorporating a reduced stress living hinge is shown in FIGS. 2 and 3. Shieldable needle assembly 12 includes a needle cannula 14 having a proximal end 16, a distal tip 18 and a lumen 19 extending therebetween. Distal tip 18 in this embodiment includes a bevel aligned at an acute angle to the longitudinal axis of needle cannula 14. Shieldable needle assembly 12 further includes a needle hub 20 which is securely and permanently engaged with proximal end 16 of needle cannula 14.

Shieldable needle assembly 12 is connected to a hypodermic syringe 22. The hypodermic syringe includes a syringe barrel 24 having opposed proximal and distal ends 26 and 28 respectively and a chamber 30 extending therebetween. Proximal end 26 of syringe barrel 24 is open and slidably receives a plunger 32. Distal end 28 of syringe barrel 24 includes an elongate tip 34 having a passage 36 extending therethrough. Distal end 28 of syringe barrel 24 preferably includes a luer collar 38 concentrically surrounding tip 34. The luer collar includes an array of internal threads which may be engaged by needle hub 20 of shieldable needle assembly 12.

Shieldable needle assembly 12 includes a needle guard 40 which comprises a cap portion 42 and a clip 44. Cap 42 is preferably molded from a thermoplastic material to include a cylindrically generated side wall 46, preferably extending circumferentially through more than 180°, and having opposed proximal and distal ends 47 and 48. Side wall 46 preferably is longer than the bevel at distal tip 18 of needle cannula 14. Cap 42 further includes an end wall 50 extending across distal end 48 of side wall 46. End wall 50 includes an aperture 51 which is slidable along needle cannula 14.

Needle guard 40 of shieldable needle assembly 12 can include clip 44. Clip 44 is retained between needle cannula 14 and cap 42. Clip 44 includes a distal wall 52 that is configured to be biased against needle cannula 14. Sufficient distal movement of cap 42 will cause distal wall 52 of clip 44 to pass beyond and biasly engage distal tip 18 of needle cannula 14, thereby preventing hinged arm assembly 54 from inadvertently returning to its retracted position and re-exposing distal tip 18. Clip 44 is preferably made of metal such as stainless steel or other material exhibiting good penetration resistance. Clip 44 generally provides more protection against penetration by distal tip 18 than provided by the plastic of cap 42 alone. Additionally, a clip made of metal or other puncture resistant material enables the plastic of the shieldable needle assembly to be selected in view of its resiliency and other performance characteristics, and without concern for the ability of the plastic to resist needle punctures.

Shieldable needle assembly 12 further includes a hinged arm assembly 54 having a proximal segment 56 and a distal segment 58, which are articulated to one another at hinge 60. Proximal segment 56 of hinged arm assembly 54 is articulated to needle hub 20 at hinge 62. Distal segment 58 of hinged arm assembly 54 is articulated to cap 42 at hinge 64, which is adjacent proximal end 47 of side wall 46. One or more of hinges 60, 62 and 64 are reduced stress living hinges as discussed above. According to one or more embodiments, the reduced stress hinges are configured such that distal segment 58, proximal segment 56 and needle guard 40 are maintained in a retracted needle tip exposed position in the absence of mechanical retention. Preferably, living hinges 60 and 62 are reduced stress hinges, and the reduced stress can be provided using the techniques described above. If the stress of hinge 62 is sufficiently reduced such that distal segment 58, proximal segment 56 and needle guard 40 can be maintained in a retracted needle tip exposed position in the absence of mechanical retention, hinges 60 and 64 can be stressed living hinges or hinge pins if desired.

Following use of syringe 22, proximal and distal segments 56 and 58 of hinged arm assembly 54 can be articulated about hinges 60, 62 and 64 toward an extended position, with needle guard 40 slidably telescoping along needle cannula 14 to substantially enclose distal tip 18. The extension of hinged arm assembly 54 from the retracted position to the extended position shown can be generated entirely from locations proximally on needle cannula 14. More particularly, proximal segment 56 of hinged arm assembly 54 extends radially outwardly from needle cannula 14 sufficiently to define a proximally facing actuation surface extending transversely from syringe barrel 24. Thus, as shown in FIG. 2, distally directed pressure by a finger of the hand engaging syringe barrel 24 will urge hinged arm assembly 54 from its retracted position to its extended position, and will thereby urge needle guard 40 toward distal tip 18 of needle cannula 14.

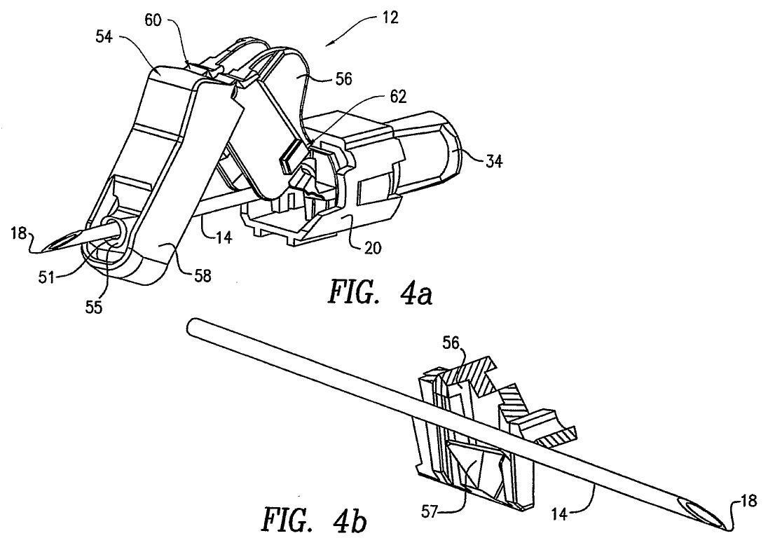

Although shown with needle guard 40 in FIGS. 2 and 3, the shieldable needle assembly described herein can be provided without a needle guard (and thus without living hinge 60). As shown in FIG. 4a, distal segment 58 can include aperture 51 at its distal end configured to slide along needle cannula 14. Aperture 51 is shown as a linear bearing 55 hingedly disposed within distal segment 58. Distally directed pressure will urge hinged arm assembly 54 from its retracted condition to its extended condition, with linear bearing 55 of distal segment 58 slidably telescoping along needle cannula 14 to substantially enclose distal tip 18. In this embodiment, as shown in FIG. 4b, proximal segment 56 preferably contains a locking mechanism 57, such as a flap lock, clip or any other suitable locking mechanism, which is configured to flexibly engage and biasly retain needle cannula 14 upon enclosure of distal tip 18 by linear bearing 55 of distal segment 58, thereby preventing hinged arm assembly 54 from inadvertently returning to its retracted position and re-exposing distal tip 18.

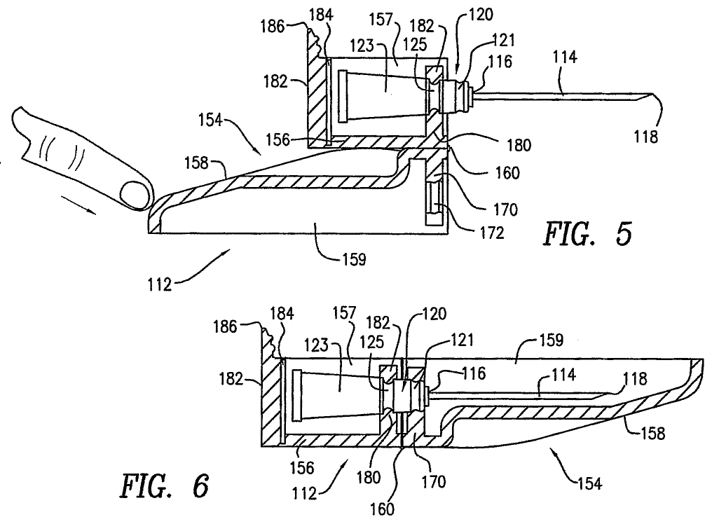

Another embodiment of a shieldable needle assembly incorporating a reduced stress living hinge is shown in FIGS. 5 and 6. Shieldable needle assembly 112 includes a needle cannula 114 having a proximal end 116 and a distal tip 118. Distal tip 118 in this embodiment includes a bevel aligned at an acute angle to the longitudinal axis of needle cannula 114. Shieldable needle assembly 112 further includes a needle hub 120 which is securely and permanently engaged with proximal end 116 of needle cannula 114. Luer hub 123 of needle hub 120 is configured to engage the internal threads of standard hypodermic syringes.

Shieldable needle assembly 112 further includes a shield body 154 having a cover 158. Cover 158 has an elongate channel 159, which extends longitudinally of shield body 154. Elongate channel 159 is closed at one end, generally U-shaped in lateral cross section along its length and has a generally flat peripheral edge generally lying in a plane and extending along both sidewalls as well as about the closed end portion. The upper surface of cover 158 is sloped toward the closed end portion with a conveniently flat surface. The resulting bevel is generally parallel to the bevel on distal tip 118 positioned within as an indicator of the needle orientation.

Cover 158 further includes a retainer 170 within elongate channel 159. In a preferred embodiment, retainer 170 includes opposed flexible fingers 172 which are engaged with anterior portion of needle hub 120 when cover 158 is closed over needle 114. A recess 121 about the anterior portion of needle hub 120 receives retainer 170. Fingers 172 may describe arcs of a circle to fit into recess 121. The fingers 172 may alternatively be sized to engage needle 114 rather than the anterior portion of needle hub 120.

Shield body 154 also includes a mount 156. Mount 156 is formed with a channel 157 aligned with the open cavity of shield body 154. Together, cover 158 and mount 156 define an elongate open cavity of the shield body 154. Mount 156 includes a socket 180 for rigidly retaining needle assembly 112. Socket 180 also includes opposed fingers 182 which extend about the waist 125 of needle hub 120 and configured to grip waist 125 more strongly than retainer 170 grips recess 121.

Mount 156 has an access end which is closed by an end piece 182. End piece 182 extends across the access end of mount 156 and is fixed thereto by a frangible sealing web 184 which extends about the end of mount 156 where the channel 157 intersects the end piece 182 in a U-shaped configuration. End piece 182 includes a finger tab 186 for gripping end piece 182 to tear sealing web 184 and fully remove end piece 182. Through the removal of end piece 182, access is provided through the access end of mount 134 to the socket of luer hub 123.

Cover 158 and mount 156 of shield body 154 are articulated to one another at reduced stress living hinge 160. As shown in FIG. 5, the stress of living hinge 160 is reduced during or after manufacture of the shield body 154 using the techniques described above. In one or more embodiments, a living hinge is provided that has substantially no internal stress such that the hinge is not urged to an open or flat configuration. In one or more embodiments, the stress in the living hinge is reduced such that cover 158 remains in a proximal needle tip exposed position in the absence of mechanical retention.

Following use of needle 114, needle cover 158 can be articulated about reduced stress living hinge 160 to substantially enclose distal tip 118. More particularly, as shown in FIG. 5, upwards directed pressure by a finger of the hand engaging needle assembly 112 will urge needle cover 158 to rotate upwardly and distally from its proximal needle tip exposed position to its distal needle tip enclosed position. As shown in FIG. 6, needle cover 158 is forced such that fingers 172 of retainer 170 engage recess 121 of needle hub 120, thereby preventing needle cover 158 from inadvertently returning to its proximal needle tip exposed position.

Although not shown in FIGS. 2-6, the shieldable needle assembly described herein can be provided with a needle cover having an elongated hollow frustoconical distal part that is similar in form and function to needle covers commonly used for protecting needles prior to use. The needle cover can also have a laterally and proximally disposed guard which acts to prevent inadvertent shield actuation before removal of the cover for use of the needle. Similar to currently available needle covers, the needle cover may be formed (e.g., injection molded) from polypropylene or other synthetic resinous material.

Thus, one or more hinges associated with a needle or shield or cover can have the internal stress reduced or completely eliminated by irradiating the entire shield or cover, or directing radiation or heat at the hinge portion during the shield manufacturing process. Such irradiation or treatment can be integrated into the manufacturing process or performed as a separate step. The desired level of stress reduction or removal will depend on the properties of the plastic material used to manufacture the shield, and the amount of spring in the hinge that can be tolerated by the particular hinge arrangement. Some hinge designs will require the stress to be completely removed from the hinge, while other designs may tolerate a certain amount of stress in the hinge.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims. For example, additional segments articulated to one another by additional living hinges (beyond those shown in FIGS. 2-6) can be included in embodiments of the shieldable needle assembly of the present invention, some or all of which are reduced stress, or exhibit substantially no internal stress, and in some embodiments, the living hinges are such that the assembly does not require mechanical retention for maintaining the shield in the needle tip exposed position. In addition, although certain shield designs with living hinges have been described herein with respect to embodiments of the invention, the invention is not intended to be limited to a particular shield design or hinge configuration.

All publications cited in the specification, both patent publications and non-patent publications, are indicative of the level of skill of those skilled in the art to which this invention pertains. All these publications are herein fully incorporated by reference to the same extent as if each individual publication were specifically and individually indicated as being incorporated by reference.

What is claimed is:

1. A shieldable needle assembly comprising:
a needle cannula having a proximal end and a distal tip; and
a safety shield comprising one or more reduced stress living hinges movable between first position in which the distal tip of the needle cannula is exposed and a second position in which the shield is configured to substantially enclose the distal tip of said needle cannula.

2. The shieldable needle assembly of claim 1, wherein the one or more reduced stress living hinges maintain the safety shield in the needle tip exposed first position in the absence of mechanical retention.

3. The shieldable needle assembly of claim 1, wherein the stress of the one or more living hinges has been reduced by heat treatment or irradiation.

4. A shieldable needle assembly comprising:
a needle cannula securely engaged with a needle hub, said needle cannula having a proximal end and a distal tip; and
a safety shield having a proximal end and a distal end, said shield further having a needle guard slidably moveable along said needle cannula and a distal segment and a proximal segment connected via a hinge, said proximal segment and said needle hub connected via a hinge and said distal segment and said needle guard connected via a hinge, said distal segment, said proximal segment and said needle guard being extensible between a retracted first position in which the distal tip of the needle cannula is exposed and an extended second position in which the needle cannula distal tip is enclosed, wherein one or more of said hinges is a reduced stress living hinge.

5. The shieldable needle assembly of claim 4, wherein the reduced stress living hinge or hinges maintain the distal segment, the proximal segment and the needle guard substantially in the retracted first position in the absence of mechanical retention.

6. The shieldable needle assembly of claim 5, wherein the stress of the living hinge or hinges has been reduced by heat treatment or irradiation.

7. The shieldable needle assembly of claim 4, wherein the needle guard comprises a clip configured to pass beyond the distal tip during extension of the shield.

8. A shieldable needle assembly comprising:
a needle cannula securely engaged with a needle hub, said needle cannula having a proximal end and a distal tip; and
a safety shield having a proximal end and a distal end, said shield further having a distal segment and a proximal segment connected via a hinge, said proximal segment and said needle hub connected via a hinge, said distal segment and said proximal segment being extensible between a retracted first position and an extended second position in which the distal tip of the needle is enclosed, said distal segment slidably moveable along said needle cannula, wherein one or both of said hinges is a reduced stress living hinge.

9. The shieldable needle assembly of claim 8, wherein the reduced stress living hinge or hinges maintain the distal segment and the proximal segment substantially in the retracted needle tip exposed first position in the absence of mechanical retention.

10. The shieldable needle assembly of claim 9, wherein the stress of the living hinge or hinges has been reduced by heat treatment or irradiation.

11. The shieldable needle assembly of claim 8, wherein the shield further comprises a linear bearing hingedly disposed within the distal segment.

12. The shieldable needle assembly of claim 8, wherein the shield further comprises a locking mechanism to lock the shield in the extended needle tip enclosed second position.

13. The shieldable needle assembly of claim 12, wherein the locking mechanism includes a flap lock configured to flexibly engage and biasly retain the needle cannula upon enclosure of the distal tip.

14. A shieldable needle assembly comprising:
a needle cannula securely engaged with a needle hub, said needle cannula having a proximal end and a distal tip; and
a safety shield having a shield body, said shield body having a cover and a mount connected via a hinge, said mount mounted to said hub, said cover moveable between a proximal first position and an distal second position to substantially enclose the distal tip of said needle cannula, wherein said hinge is a reduced stress living hinge.

15. The shieldable needle assembly of claim 14, wherein the reduced stress living hinge maintains the cover in the proximal needle first position in the absence of mechanical retention.

16. The shieldable needle assembly of claim 15, wherein the stress of the living hinge has been reduced by heat treatment or irradiation.

17. The shieldable needle assembly of claim 14, wherein the cover comprises a retainer engagable with the needle hub upon enclosure of the distal tip.

18. A method of making a shielded needle assembly comprising:
providing a foldable shield having at least one living hinge between two folded segments, the living hinge exhibiting internal stress such that the folded segments are urged to an open or flat configuration, assembling the shield to a device including a needle cannula including a distal tip such that one of the folded segments is configured to cover the distal tip of the needle, exposing at least the living hinge to heat and/or irradiation sufficient to remove substantially all of the internal stress in the living hinge.

19. The method of claim 18, further comprising irradiating or heat treating the shield to sterilize the shield and irradiating and/or heat treating at least the living hinge with an additional amount of heat and/or irradiation for a period of time sufficient to remove substantially all of the internal stress of the living hinge.
